(19) United States
(12) Patent Application Publication
(10) Pub. No.: US 2025/0391537 A1
(43) Pub. Date: Dec. 25, 2025

(54) MASSAGE MACHINE AND MASSAGE SYSTEM

(71) Applicant: FUJI MEDICAL INSTRUMENTS MFG. CO., LTD., Osaka (JP)

(72) Inventors: Yasuki MAKISHI, Osaka (JP); Mitsuaki FUJISHIRO, Osaka (JP)

(73) Assignee: FUJI MEDICAL INSTRUMENTS MFG. CO., LTD., Osaka (JP)

(21) Appl. No.: 19/215,213

(22) Filed: May 21, 2025

(30) Foreign Application Priority Data
Jun. 21, 2024 (JP) 2024-100590
Feb. 10, 2025 (JP) 2025-020316

(51) Int. Cl. G16H 20/40; A61H 7/00

(52) U.S. Cl. CPC G16H 20/40; A61H 7/007; A61H 2201/501; A61H 2201/5048

(57) ABSTRACT

Provided are a massage machine and a massage system that perform test treatments respectively to a plurality of treatment target portions of a user, catch a result of the test treatments by communicating with the user, and determine a treatment course composed of a combination of a plurality of massage manipulations by the treatment parts based on the result of the test treatments caught by the communication with the user.

### CROSS-REFERENCE TO RELATED APPLICATION

This application claims priority to Japanese Patent Application Nos. 2024-100590, filed on Jun. 21, 2024, and 2025-20316, filed on Feb. 10, 2025, the disclosures of which are incorporated herein by reference in its entirety.

### FIELD OF THE INVENTION

The present disclosure relates to a massage machine for treating a user, and a massage system.

### BACKGROUND OF THE INVENTION

Conventionally, there has been known a chair-type massage machine which treats each part of a user with a treatment element such as a kneading ball or an airbag with the user being seated (JP 2024-26638 A).

In this massage machine, a plurality of treatment courses each composed of a combination of a plurality of massage manipulations (e.g., kneading up and kneading down by a kneading ball, pinching and pushing up by an airbag, and the like) by treatment elements, and the user selects and executes a treatment course by an operation unit such as a remote control part in accordance with his or her body conditions.

In the above-described massage machine, the user selects the treatment course by himself/herself, but there is a case where the selected treatment course is not suitable for the user.

### SUMMARY OF THE INVENTION

An object of the present disclosure is to provide a massage machine and a massage system that are able to properly treat a user.

The following presents a simplified summary of the disclosure disclosed herein in order to provide a basic understanding of some aspects of the disclosure. This summary is not an extensive overview of the disclosure. It is intended to neither identify key or critical elements of the disclosure nor delineate the scope of the disclosure. Its sole purpose is to present some concepts of the disclosure in a simplified form as a prelude to the more detailed description that is presented later.

A massage machine according to the present disclosure includes treatment parts for treating a plurality of treatment target portions of a user, a voice output part, a voice input part, and a control part to which the treatment parts, the voice output part, and the voice input part are connected, the control part performing: a step of performing test manipulations using the treatment parts respectively to the plurality of target portions of the user; a step of catching a result of the test treatments by communicating with the user through the voice output part and the voice input part; and a step of determining a treatment course composed of a plurality of a combination of massage manipulations by the treatment parts based on the result of the test treatments caught by the communication with the user.

It can be configured such the control part performs, before performing the test treatments, a step of performing communication for test preparation for catching at least one of activities and conditions of the user by communicating with the user through the voice output part and the voice input part, and determines a treatment target portion to which each corresponding one of the test treatments is performed based on the result of the communication for test preparation.

It can be configured such that the massage machine further includes a storing part that is connected to the control part and stores a plurality of massage manipulations be respectively performed by the treatment parts, in which the treatment course is composed of the massage manipulations stored in the storing part.

The massage machine can be configured such that in the step of determining the treatment course, the control part selects a certain massage manipulation or certain massage manipulations from the plurality of massage manipulations stored in the storing part based on the result of the test treatments caught by the communication with the user, and composes the treatment course by the selected massage manipulation or massage manipulations.

The massage machine can be configured such that the storing part stores at least one basic set composed of a combination of certain massage manipulations, and the control part composes the treatment course based on the plurality of massage manipulations and the at least one basic set stored in the storing part.

A massage system of the present disclosure includes: treatment parts for treating a plurality of treatment target portions of a user, a voice output part, a voice input part, a machine control part to which the treatment parts, the voice output part, and the voice input part are connected, and a communication part; and a server including a server control part and being able to communicate with the massage machine, in which the machine control part is configured to perform test treatments respectively to the plurality of treatment target portions of the user using the treatment parts, and the server control part is configured to perform a step of catching a result of the test treatments by communicating with the user through the voice output part and the voice input part of the massage machine, and a step of determining a treatment course composed of a combination of a plurality of massage manipulations by the treatment parts based on the result of the test treatments caught by the communication with the user.

### BRIEF DESCRIPTION OF THE DRAWINGS

The foregoing and other features of the present disclosure will become apparent from the following description and drawings of an illustrative embodiment of the disclosure.

FIG. 1 is a perspective view of a massage machine according to the present embodiment.

FIG. 2 is a view showing a configuration of a massage unit included in the massage machine.

FIG. 3 is a view for explaining a flow of an automatic massage in the massage machine.

FIG. 4 is a view showing a first table and a course menu stored in a storing part of the massage machine.

FIG. 5 is a view showing a second table and a course menu stored in the storing part of the massage machine.

FIG. 6 is a view showing a third table and a course menu stored in the storing part of the massage machine.

FIG. 7 is a view showing a fourth table and a course menu stored in the storing part of the massage machine.

FIG. 8 is a view showing a fifth table and a course menu stored in the storing part of the massage machine.

FIG. 9 is a view showing a sixth table and a course menu stored in the storing part of the massage machine.

FIG. 10 is a view showing a seventh table and a course menu stored in the storing part of the massage machine.

FIG. 11 is a view showing an eighth table and a course menu stored in the storing part of the massage machine.

FIG. 12 is a view showing a ninth table and a course menu stored in the storing part of the massage machine.

FIG. 13 is a view showing a view for explaining a configuration of a massage system including a massage machine and a server.

### DETAILED DESCRIPTION OF THE PREFERRED EMBODIMENT

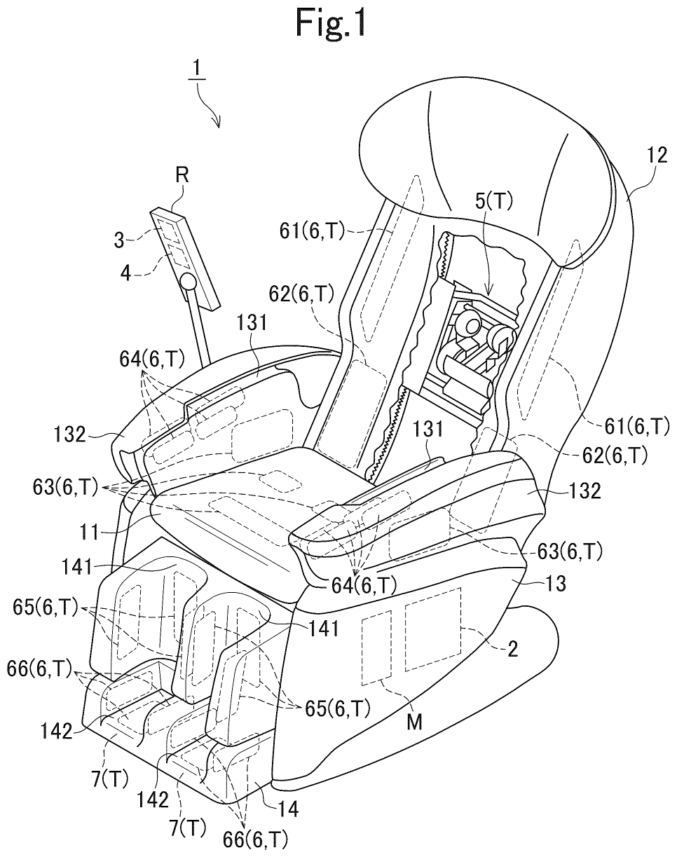

Hereinafter, a massage machine according to an embodiment of the present disclosure will be described with reference to FIG. 1 to FIG. 12. In the following description, the front-rear direction, the right-left direction, and the vertical direction are the front-rear direction, the right-left direction, and the vertical direction with respect to the user when the user is seated on the massage machine in a normal posture.

As shown in FIG. 1, a massage machine 1 according to this embodiment is a chair-type massage machine, and includes treatment parts T for treating treatment target portions (portions to be treated) of a user, a voice output part 3, a voice input part 4, and a control part 2 to which the treatment parts T, the voice output part 3, and the voice input part 4 are connected.

Specifically, the massage machine 1 includes a seat part 11, a backrest part 12 disposed on the rear side of the seat part 11, armrest parts 13 disposed respectively on both the right and left sides of the seat part 11, an ottoman 14 disposed on the front side of the seat part 11, and a base that serves as a support for these parts. The massage machine 1 also includes the control part 2 that controls each component of the massage machine 1, and the voice output part 3 and the voice input part 4 that are connected to the control part 2. The massage machine 1 of this embodiment includes a remote control part (operation part) R for operating the massage machine 1, and the remote control part R incorporates the voice output part 3 and the voice input part 4. The massage machine 1 of this embodiment includes a storing part M that is connected to the control part 2 and stores a plurality of massage manipulations to be performed by the treatment parts T.

In the massage machine 1 of this embodiment, the treatment parts T include a massage unit 5 disposed in the backrest part 12, airbags (treatment elements) 6 disposed in the seat part 11, the backrest part 12, the armrest parts 13, the ottoman 14, etc., and sole rollers (treatment elements) 7 disposed in the ottoman 14. A detailed description will be given in detail on the components 5, 6, 7 included in these treatment parts T.

The base is disposed below the seat part 11 to serve as a support for the components 11 to 14. In FIG. 1, the base is covered by the seat part 11, the backrest part 12, the armrest parts 13, and the ottoman 14, and therefore is not visible from the outside.

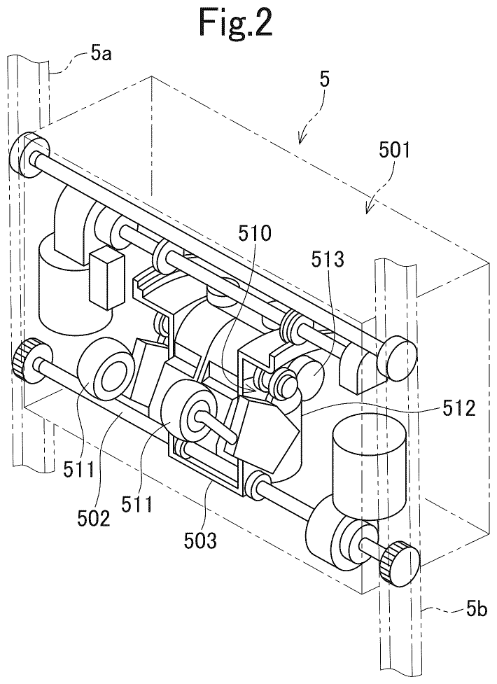

The backrest part 12 is disposed so as to be tiltable relative to the seat part 11, and is tilted by an actuator for the rotation of the backrest part 12. As shown in FIG. 2, disposed inside the backrest part 12 are the massage unit 5 and a pair of guide rails 5a, 5b that guide the massage unit 5 in the vertical direction along the backrest part 12.

The pair of guide rails 5a, 5b are disposed with a space therebetween in the right-left direction, and the pair of guide rails 5a, 5b extend in the vertical direction along the backrest part 12.

The massage unit 5 includes a pair of right and left massage balls (treatment elements) 511, and is configured to perform various massages (mechanical massages) using the pair of massage balls 511. The massage unit 5 is movable in the vertical direction along the pair of guide rails 5a, 5b.

Specifically, the massage unit 5 includes a rectangular main frame 501, a lifting drive shaft 502 disposed on the main frame 501, a swing frame 503 mounted to the lifting drive shaft 502 so as to be freely swingable in the forward and backward direction, a treatment element drive unit 510 disposed on the swing frame 503, and a mechanism for advancing and retreating the swing frame 503 (treatment element drive unit 510) in the forward and backward direction.

The treatment element drive unit 510 includes a pair of right and left massage balls (treatment elements) 511, a massage mechanism for performing massaging motions by eccentrically rotating the massage balls 511, and a tapping mechanism for performing tapping motions by swinging the massage balls 511 in the forward and backward direction. The massaging mechanism includes a motor 512 for massaging as an actuator (drive source), and the tapping mechanism includes a motor 513 for tapping as an actuator (driving source).

The armrest parts 13 each include an arm unit 132 provided with a forearm accommodation recess 131 for accommodating the forearm of the user.

The ottoman 14 is disposed to be rotatable around a rotating axis extending in the right and left direction disposed near a front end part of the seat part 11 and is configured to be rotated by an actuator that rotates the ottoman 14. The ottoman 14 of this embodiment is rotatable between an upright position and a horizontal position. The ottoman 14 includes leg accommodation recess parts 141 and foot accommodation recess parts 142 respectively accommodating the right and left legs and feet of the user. Sole rollers 7 are disposed in bottom parts of the foot accommodation recess parts 142, and are configured to massage the soles by reciprocal motions in the forward and backward direction.

The seat part 11, the backrest part 12, the armrest parts 13, and the ottoman 14 respectively have airbags 6 mounted inside thereof. Each of the airbags 6 is expanded and contracted by air feeding and discharging so as to perform an air massage for each of the treatment target portions of the user.

Specifically, the airbags 6 include shoulder-arm airbags 61 for massaging the upper arms, lower-back airbags 62 for massaging the lower back, seating part airbags 63 for massaging the thighs, forearm airbags 64 for massaging the forearms and the hands, calf airbags 65 for massaging the calves, and sole airbags 66 for massaging the soles.

The shoulder-arm airbags 61 are disposed respectively at center parts in the vertical direction on both right and left sides of the backrest part 12. The lower-back airbags 62 are disposed respectively on both the right and left sides of a lower part of the backrest part 12. The seating part airbags 63 are disposed respectively in the seat part 11, and lower parts of the arm units 132 of the armrest parts 13. The forearm airbags 64 are disposed respectively on both the lateral sides of the forearm accommodation recesses 131. The calf airbags 65 are disposed respectively on both the lateral sides of the leg accommodation recess parts 141 of the ottoman 14, and bottom sides of each of the leg accommodation recess parts 141. The sole airbags 66 are disposed respectively on both the lateral sides and the bottom sides of each of the foot accommodation recess parts 142. The sole airbags 66 are disposed respectively on both the lateral sides and the bottom sides of each of the foot accommodation recess parts 142.

The control part 2 is composed of a microcomputer including, for example, a CPU (Central Processing Unit), a non-volatile memory element such as a ROM (Read Only Memory) or an EEPROM (Electrically Erasable Programmable Read Only Memory) that stores in advance various programs executed by the CPU and data necessary for their execution, a volatile memory element such as a RAM (Random Access Memory) that serves as a so-called working memory of the CPU, and their peripheral circuits. When an automatic massage is selected by operating the remote control part R, the control part 2 communicates with the user through the voice output part 3 and the voice input part 4, creates a treatment course suitable for the user based on this communication, and causes the treatment part T to execute this treatment course.

The above communication is not limited to voice, but includes chat through text input (communication through text). The control part 2 is configured to display an input screen, through which text can be inputted, on a display part of the remote control part R or the like. The user can perform the above communication by inputting into the input screen. Further, the above communication can be performed through a portable terminal which the user uses. That is, the control part 2 can be configured to display the input screen on the display part of the portable terminal through short-range wireless communication or the like, and obtain the input content (communication content) input into the input screen by the user.

The control part 2 of this embodiment is equipped with, for example, AI (artificial intelligence) and communicates with the user to obtain information (keywords or the like) required to create a treatment course. Details of creating a treatment course based on this communication will be described later.

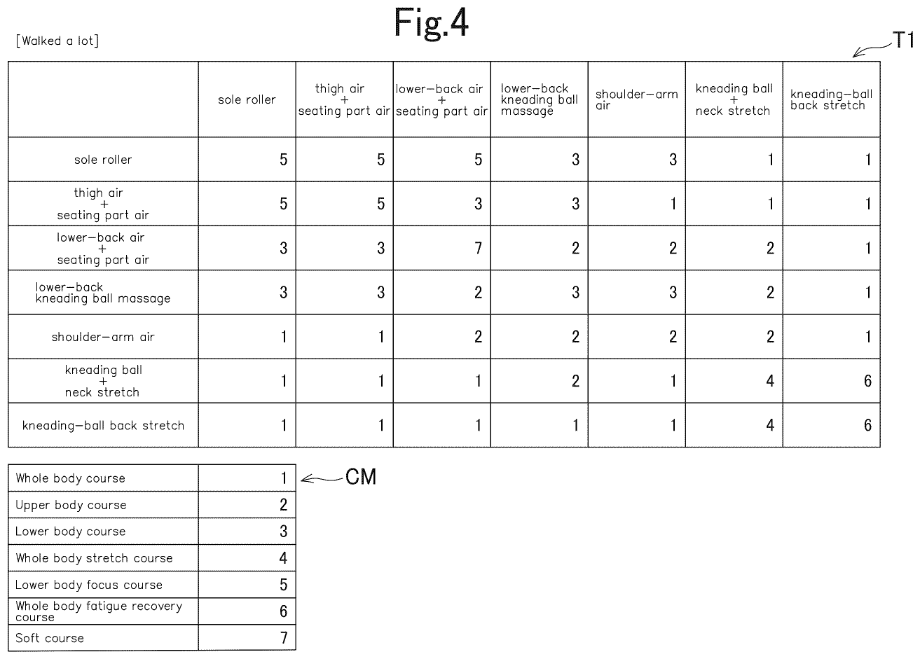

A memory part M is a storage device with a relatively large capacity, such as a hard disk drive (HD) or a solid state drive (SSD), and stores various data or the like used for processing in the control part 2. The memory part M of this embodiment stores at least information on the massage manipulations to be performed by the treatment parts T, the first to ninth tables T1 to T9 (see FIG. 4 to FIG. 12), and course menus CM (see FIG. 4 to FIG. 12), or the like.

In the massage machine 1 configured as described above, when the remote control part R is operated and an automatic massage button is pressed (turned ON), a treatment course suitable for physical conditions of the user is created based on the communication with the user through the voice output part 3 and the voice input part 4, and this created treatment course is implemented.

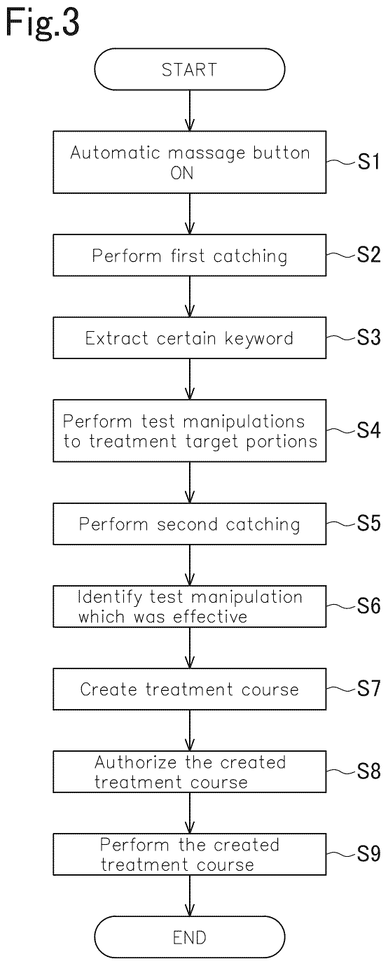

Specific examples of the created treatment course based on the communication with the user will be described with reference to FIG. 3 to FIG. 12.

As shown in FIG. 3, an automatic massage button of an operation part (operation remote control or the like) of the massage machine 1 is pressed by the user who sits on the massage machine 1 (Step S1), then the control part 2 catches the user's physical activities of the day up to the sitting on the massage machine 1 (first catching) by communicating with the user through the voice output part 3 and the voice input part 4 (Step S2), and then a certain keyword (set keyword) on the user's physical activities from the communication with the user (first catching) is extracted (Step S3).

The certain keyword in this embodiment is a keyword for identifying which part of the user's body is fatigued and how it is fatigued (i.e., what massage manipulation should be performed on which part (test manipulation)), and includes, for example, nine keywords: “walked a lot,” “talked a lot,” “feeling good,” “did physical exercise,” “worked desk job,” “kept quiet,” “spent with high stress,” “did not physical exercise,” and “tired from sleeping,” but are not limited to these nine keywords. The nine keywords in this embodiment are examples of the causes affecting on stiff shoulders or the like, among the places where the user stayed for a certain period of time and their condition at that time. In addition, these keywords are items selected so as to enable linkage with data such as the range of movement and means of movement on daily basis, or data using health care applications.

Unless the control part 2 is able to extract a certain key word at the first catching, the user may input into the remote control part R the content corresponding to any one of the above nine key words, or select a button corresponding to any key word of the above nine key words by operating the remote control part R, so that the control part 2 extracts or obtains a certain key word.

Subsequently, the control part 2, based on the key word extracted at Step S3, performs test manipulations using the treatment parts T respectively to treatment target portions of the user, that is, perform a test treatment (Step S4). This test manipulation is a massage manipulation (treatment) performed for a short period of time (a time of period shorter than the time of the treatment course created after a later described second catching) before creating the treatment course. Each test manipulation is performed to each of the plurality of treatment target portions selected based on the key word extracted at the first catching.

When each of the test manipulations is completed, the control part 2 catches a test manipulation which was effective in the test manipulations implemented (second catching) by communicating with the user through the voice output part 3 and the voice input part 4 (Step S5). The control part 2 identifies a test manipulation which was effective in the test manipulations at the second catching (Step S6), and creates a treatment course composed of a combination of a plurality of massage manipulations (Step S7). In the massage machine 1 of this embodiment, the control part 2 identifies a test manipulation which appeared to be effective based on the second catching which area (treated area) was painful when the test manipulations were performed and which manipulation the user would like to have performed again.

Subsequently, the control part 2 causes the remote control part R to display a created treatment course (or information on a treatment course), so that the user is allowed to input authorization for this created treatment course (press an authorization button or the like) (Step S8). Then, the created treatment course is implemented (Step S9).

A more specific description will be given on Step S2 to Step S7 in the flow of creating the aforementioned treatment course.

The control part 2 communicates with the user so as to be able to extract any of nine items (nine keywords): “walked a lot,” “talked a lot,” “feeling good,” “did physical exercise,” “worked desk job,” “kept quiet,” “spent with high stress,” “did not physical exercise”, and “tired from sleeping” by raising a question such as “where did you go” or “what job did you do”.

When the control part 2 extracts a keyword, for example, “walked a lot” in the first catching, the control part 2 implements “sole roller”, “thigh air+seating part air”, “lower-back air+seating part air”, “lower-back kneading ball massage”, “shoulder-arm air”, “kneading ball+neck stretch”, and “kneading-ball back stretch” that are test manipulations in a first table T1 (see FIG. 4) stored in a storing part M and associated with the keyword “walked a lot”.

Here, the “sole roller” refers to a manipulation of massaging the soles of the user by the sole rollers 7 disposed in the ottoman 14. The “thigh air+seating part air” refers to a manipulation of massaging the thighs and the calves of the user by the calf airbags 65 and the seating part airbags 63. The “lower-back air+seating part air” refers to a manipulation of massaging the lower back and the thighs of the user by the lower-back airbags 62 and the seating part airbags 63. The “lower-back kneading ball massage” refers to a manipulation of massaging the lower back of the user by the massage balls 511 of the massage unit 5. The “shoulder-arm air” refers to a manipulation of massaging the upper arms of the user by the shoulder-arm airbags 61. The “kneading ball+neck stretch” refers to a massage of pressing the neck and shoulder upward by the massage balls (treatment elements) 511 of the massage unit 5, and massaging the neck and shoulder and stretching the neck of the user by tilting the backrest part 12 rearward relative to the seat part 11. The “kneading-ball back stretch” refers to a massage of pressing upward the back of the user by the massage balls 511 (treatment elements) of the massage unit 5, and stretching the back of the user by tilting the backrest part 12 rearward relative to the seat part 11.

After the completion of the test manipulations, the control part 2 communicates with the user so as to be able to identify (that is, catch from the user) a test manipulation which was effective in the test manipulations at the second catching of Step S5, and creates a treatment course at Step S7 based on the test manipulation(s) identified by the second catching at Step S6, and the first table T1 and a course menu CM (see FIG. 4) stored in the storing part M. The course menu CM of this embodiment includes (1. Whole body course, 2. Upper body course, 3. Lower body course, 4. Whole body stretch course, 5. Lower body focus course, 6. Whole body fatigue recovery course, 7. Soft course). Each of these massage courses are composed of a combination of manipulations of massages which are selected and combined based on the past knowledge or the like so as to be able to effectively treat each of the treatment target portions where fatigue or the like accumulates. These massage courses are set in advance.

When the control part 2 identifies two test manipulations at the second catching (e.g., “lower-back kneading ball massage” and “sole roller”), the control part 2 creates the lower body course as the treatment course from the numerical figure “3” at the crossing position of the row of “lower-back kneading ball massage” and the column of “sole roller” in the first table T1, and the column corresponding to “3” of the course menu CM.

When the control part 2 identifies one test manipulation at the second catching (e.g., “sole roller”), the control part 2 creates the lower body focus course as the treatment course from the numerical figure “5” at the crossing position of the row of “sole roller” and the column of “sole roller” in the first table T1, and the column corresponding to “5” of the course menu CM.

When the control part 2 identifies three test manipulations at the second catching (e.g., “sole roller”, “thigh air+seating part air”, “shoulder-arm air”), the control part 2 creates a plurality of groups each composed of two manipulations (e.g., a group of “sole roller” and “thigh air+seating part air”, a group of “thigh air+seating part air” and “shoulder-arm air”), and determines “5” and “1” from these groups of manipulations and the first table T1. Then, the control part 2 creates “lower body focus course” and “whole body course”, and integrates these created “lower body focus course” and “whole body course” to create the treatment course.

In this integration, if the created two courses are, for example, the same course, they are determined as one course, and if they are different courses, they are integrated into one course, or the control part 2 prompts the user to select any one of the courses by an operation remote control or the like. The integration can be configured such that overlapping manipulations in the two courses are grouped to create a new course. If the treatment areas of the manipulations of the two different courses are close to each other, the treatment areas can be expanded and integrated.

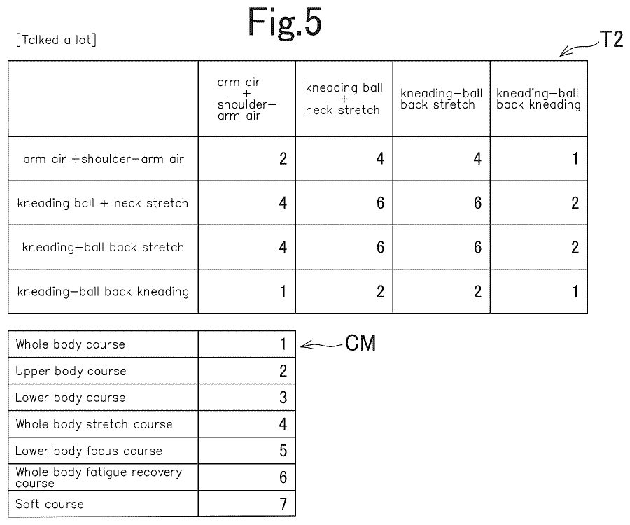

When the control part 2 extracts a keyword, for example, “talked a lot”, the control part 2 implements “arm air”+“shoulder-arm air”, “kneading ball+neck stretch”, “kneading-ball back stretch”, and “kneading-ball back kneading” that are test manipulations in a second table T2 (see FIG. 5) stored in the storing part M and associated with the keyword “talked a lot”.

Here, the “arm air” refers to a manipulation of massaging the forearms of the user by the forearm airbags 64. The “kneading ball+back kneading” refers to a manipulation of massaging the back of the user by the massage balls 511 (treatment elements) of the massage unit 5.

After the completion of the test manipulations, the control part 2 communicates with the user so as to be able to identify a test manipulation which was effective in the test manipulations at the second catching of Step S5, and creates a treatment course at Step S7 based on the test manipulation(s) identified by the second catching at Step S6, and the second table T2 and the course menu CM (see FIG. 5) stored in the storing part M.

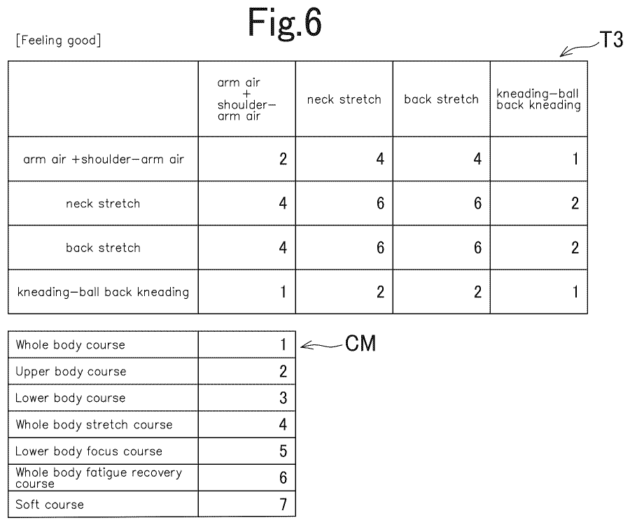

When the control part 2 extracts a keyword, for example, “feeling good”, the control part 2 implements “arm air+shoulder-arm air”, “neck stretch”, “back stretch”, and “kneading-ball back kneading” that are test manipulations in a third table T2 (see FIG. 6) stored in the storing part M and associated with the keyword “feeling good”.

The “back stretch” refers to a manipulation of stretching the back of the user by tilting the backrest part 12 backward relative to the seat part 11.

After the completion of the test manipulations, the control part 2 communicates with the user so as to be able to identify a test manipulation which was effective in the test manipulations at the second catching of Step S5, and creates a treatment course at Step S7 based on the test manipulation(s) identified by the second catching at Step S6, and the third table T3 and the course menu CM (see FIG. 6) stored in the storing part M.

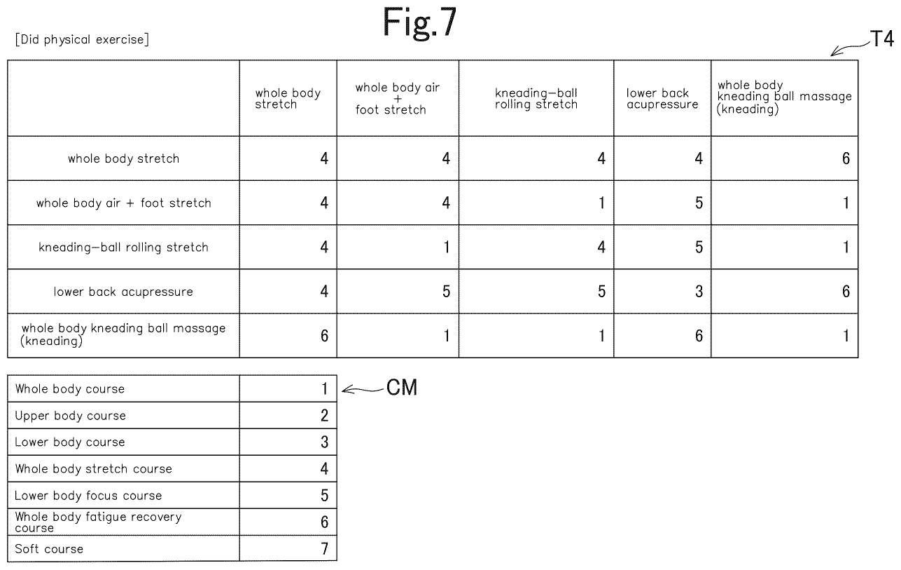

When the control part 2 extracts a keyword, for example, “did physical exercise” at the first catching, the control part 2 implements “whole body stretch”, “whole body air+foot stretch”, “kneading-ball rolling stretch”, “lower back acupressure”, and “whole body kneading ball massage” that are test manipulations in a fourth table T4 (see FIG. 7) stored in the storing part M and associated with the keyword “did physical exercise”.

Here, the “whole body stretch” refers to a manipulation composed of a combination of back stretch of stretching by pressing back muscles upward using the massage balls (treatment elements) 511, lower-back stretch of stretching by pressing lower back muscles using the lower-back air, and neck stretch of stretching neck muscles using the massage balls (treatment elements) 511. The “whole body air+foot stretch” refers to a manipulation of massaging the whole body and the legs of the user by extending the ottoman 14 forward with the upper body held by the airbags 61 to 64 for the upper body and with the legs held by the airbags 65 for the lower legs provided in the ottoman 14. The legs can be further stretched by additionally rotating the ottoman 14. The “kneading-ball rolling stretch” refers to a stretching manipulation of pressing upward the back muscles, the buttock muscles, and the leg muscles while rotating the kneading balls (treatment elements) 511. The “lower back acupressure” is a manipulation of performing acupressure on the lower back of the user by the massage balls (treatment elements) 511 of the massage unit 5. The “whole body kneading ball massage” refers to a manipulation of massaging an area from the lower back to the shoulders by the massage balls (treatment elements) 511 of the massage unit 5 while vertically moving the massage unit 5 along the pair of guide rails 5a, 5b.

After the completion of the test manipulations, the control part 2 communicates with the user so as to be able to identify a test manipulation which was effective in the test manipulations at the second catching of Step S5, and creates a treatment course at Step S7 based on the test manipulation(s) identified by the second catching at Step S6, and the fourth table T4 and the course menu CM (see FIG. 7) stored in the storing part M. The timing of the second catching is not limited to the timing after the completion of the test manipulations. The second catching can be performed during the test manipulations are performed. For example, the second catching can be performed based on a keyword such as “painful” or “comfortable” which the user utters during the test manipulations.

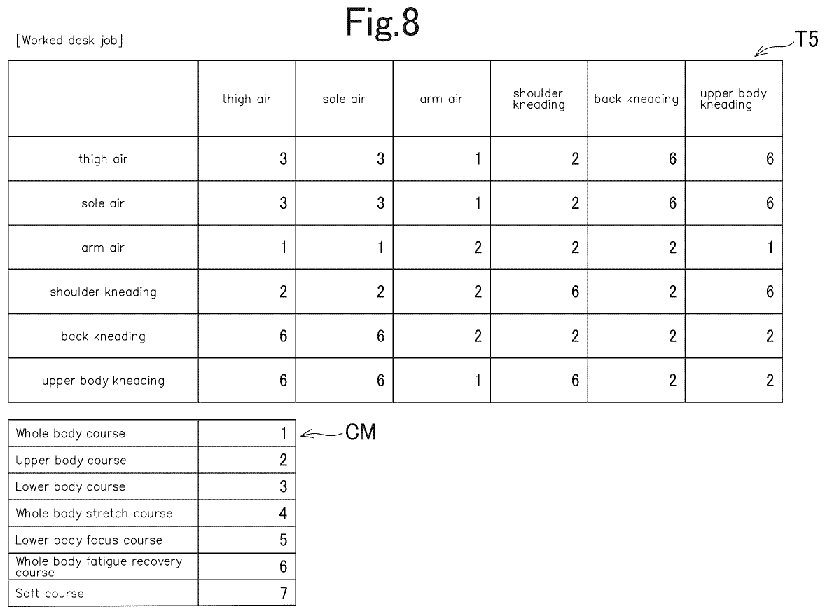

When the control part 2 extracts a keyword, for example, “worked desk job”, the control part 2 implements “thigh air”, “sole air”, “arm air”, “shoulder kneading”, “back kneading”, and “upper body kneading” that are test manipulations in a fifth table T5 (see FIG. 8) stored in the storing part M and associated with the keyword “worked desk job”.

Here, the “sole roller” refers to a manipulation of massaging the soles of the user by the sole airbags 66. The “shoulder kneading”, “back kneading”, and the “upper body kneading” are manipulations of kneading the shoulders, the back, and the upper body by the massage balls (treatment elements) 511 of the massage unit 5.

After the completion of the test manipulations, the control part 2 communicates with the user so as to be able to identify a test manipulation which was effective in the test manipulations at the second catching of Step S5, and creates a treatment course at Step S7 based on the test manipulation(s) identified by the second catching at Step S6, and the fifth table T5 and the course menu CM (see FIG. 8) stored in the storing part M.

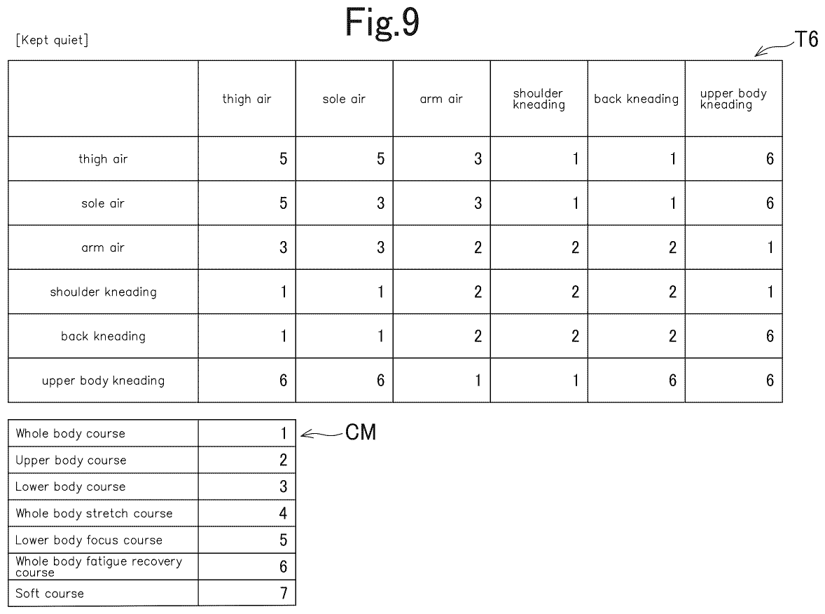

When the control part 2 extracts a keyword, for example, “kept quiet”, the control part 2 implements “thigh air”, “sole air”, “arm air”, “shoulder kneading”, “back kneading”, and “upper body kneading” that are test manipulations in a sixth table T6 (see FIG. 9) stored in the storing part M and associated with the keyword “kept quiet”.

After the completion of the test manipulations, the control part 2 communicates with the user so as to be able to identify a test manipulation which was effective in the test manipulations at the second catching of Step S5, and creates a treatment course at Step S7 based on the test manipulation(s) identified by the second catching at Step S6, and a sixth table T6 and a course menu CM (see FIG. 9) stored in the storing part M.

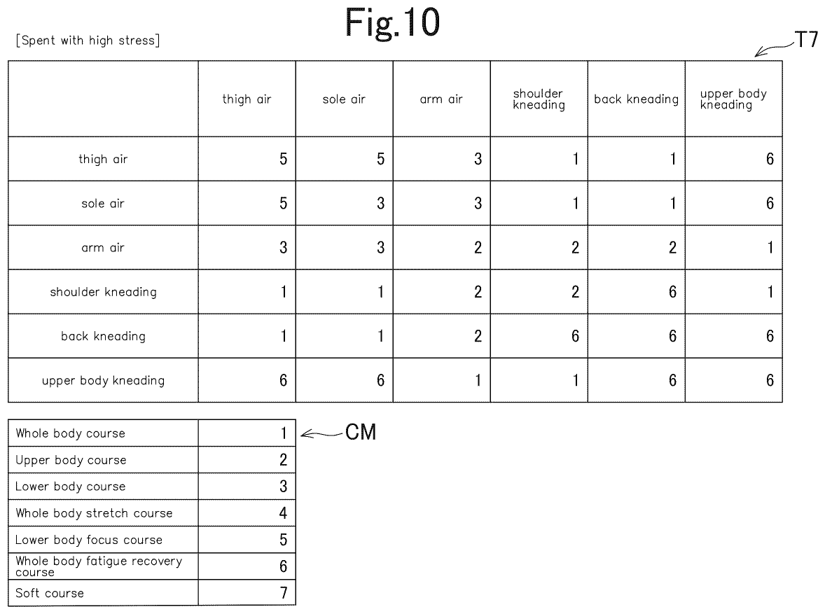

When the control part 2 extracts a keyword, for example, “spent with high stress”, the control part 2 implements “thigh air”, “sole air”, “arm air”, “shoulder kneading”, “back kneading”, and “upper body kneading” that are test manipulations in a seventh table T7 (see FIG. 10) stored in the storing part M and associated with the keyword “spent with high stress”.

After the completion of the test manipulations, the control part 2 communicates with the user so as to be able to identify a test manipulation which was effective in the test manipulations at the second catching of Step S5, and creates a treatment course at Step S7 based on the test manipulation(s) identified by the second catching at Step S6, and the seventh table T7 and the course menu CM (see FIG. 10) stored in the storing part M.

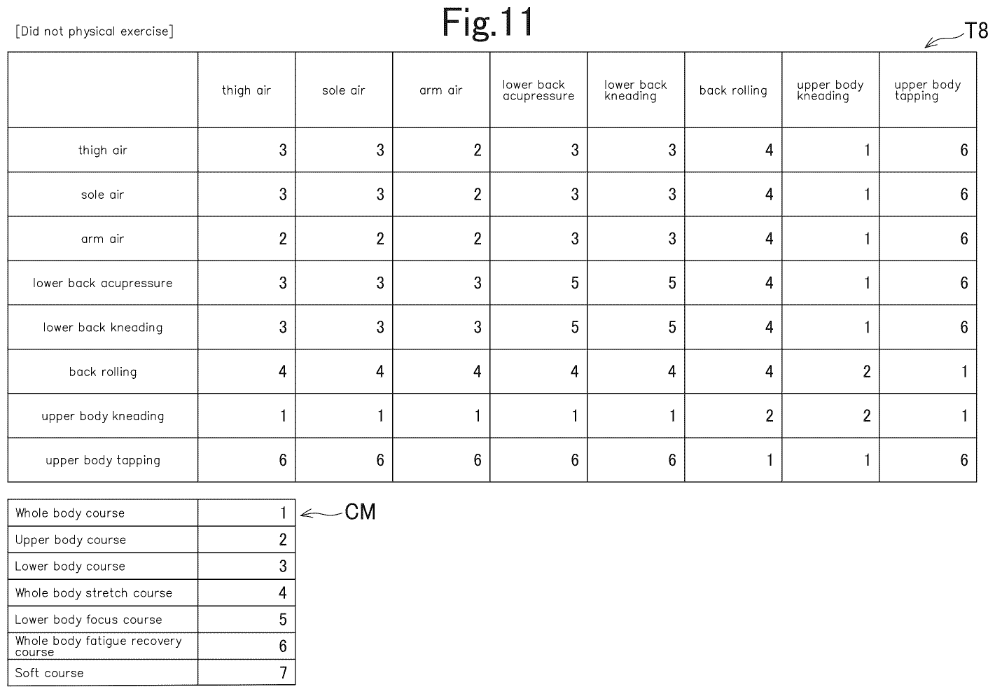

When the control part 2 extracts a keyword, for example, “did not physical exercise”, the control part 2 implements “thigh air”, “sole air”, “arm air”, “lower back acupressure”, “lower back kneading”, “back rolling”, “upper body kneading”, and “upper body tapping” that are test manipulations in an eighth table T8 (see FIG. 11) stored in the storing part M and associated with the keyword “did not physical exercise”.

Here, the “back rolling” refers to a manipulation of massaging the back of the user by vertically moving the massage unit 5 while rolling the massage balls (treatment elements) 511 of the massage unit 5. The “upper body tapping” is a manipulation of massaging the upper body of the user by vertically moving the massage unit 5 while vibrating the massage balls (treatment elements) 511 of the massage unit 5 in the front-rear direction.

After the completion of the test manipulations, the control part 2 communicates with the user so as to be able to identify a test manipulation which was effective in the test manipulations at the second catching of Step S5, and creates a treatment course at Step S7 based on the test manipulation(s) identified by the second catching at Step S6, and the eighth table T8 and the course menu CM (see FIG. 11) stored in the storing part M.

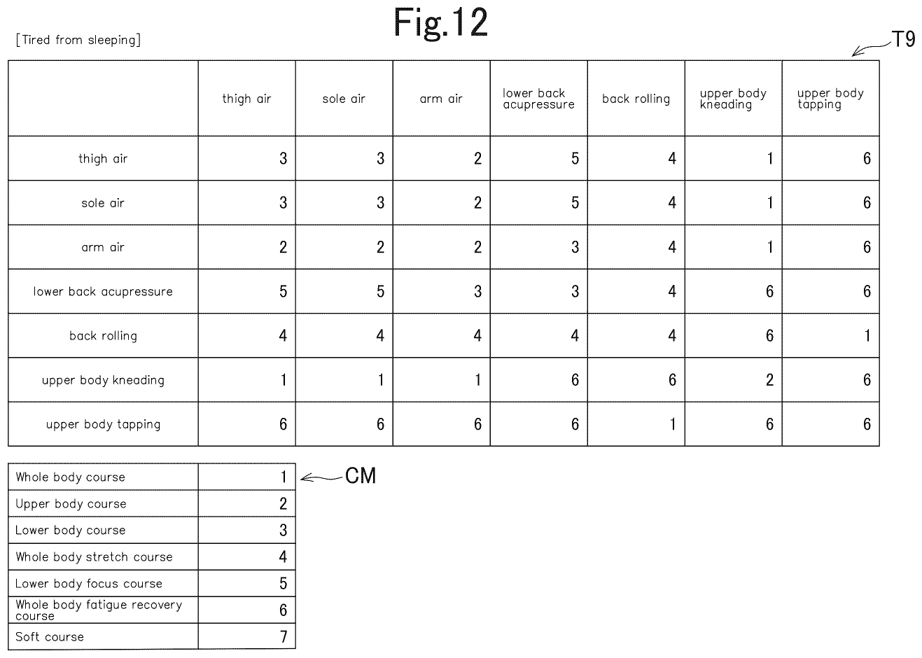

When the control part 2 extracts a keyword, for example, “tired from sleeping”, the control part 2 implements “thigh air”, “sole air”, “arm air”, “lower back acupressure”, “back rolling”, “upper body kneading”, and “upper body tapping” that are test manipulations in a ninth table T9 (see FIG. 12) stored in the storing part M and associated with the keyword “tired from sleeping”.

After the completion of the test manipulations, the control part 2 communicates with the user so as to be able to identify a test manipulation which was effective in the test manipulations at the second catching of Step S5, and creates a treatment course at Step S7 based on the test manipulation(s) identified by the second catching at Step S6, and the ninth table T9 and the course menu CM (see FIG. 12) stored in the storing part M.

The massage machine 1 mentioned above includes the treatment parts for treating a plurality of treatment target portions of a user, the voice output part 3, the voice input part 4, and the control part 2 to which the treatment parts T, the voice output part 3, and the voice input part 4 are connected. The control part 2 performs: Step S4 of performing test manipulations (test treatments) using the treatment parts T respectively to the plurality of treatment target portions of the user; Step S5 of catching a result of the test manipulations by communicating with the user (second catching) through the voice output part 3 and the voice input part 4; and Step S7 of creating (determining) a treatment course composed of a combination of a plurality of massage manipulations by the treatment parts T based on the result of the test manipulations (test treatments) caught at the second catching.

Thus, a proper treatment is applied to the user at the time of the treatment because a treatment course is created (determined) based on the content acquired by catching of the result of the test manipulations applied to the respective treatment target portions by the treatment parts T (e.g., comfort with the test manipulations applied to the respective treatment target portions) at the second catching.

In the massage machine 1 of this embodiment, the control part 2 performs, before Step S4 for performing the test manipulations (test treatments), Step S2 of performing the first catching (communication for test preparation) for catching at least one of activities and conditions of the user by communicating with the user through the voice output part 3 and the voice input part 4, and determines a treatment target portion to which each corresponding one of the test manipulations is applied based on the first catching.

According to this configuration, the number of portions to which test manipulations are applied can be limited by the first catching, and thereby a treatment course suitable for the user can be efficiently determined.

The massage machine 1 of this embodiment further includes the storing part M that is connected to the control part 2 and stores a plurality of massage manipulations to be respectively performed by the treatment parts T, in which a treatment course is composed of a massage course (massage manipulation) stored in the storing part M. Thus, the configuration that a plurality of massage courses are stored in the storing part M connected to the control part 2 enables the control part 2 to instantly determine a treatment course based on the second catching.

The massage machine of the present disclosure is not limited to the above-mentioned embodiment, and various modifications can be made without departing from the scope of the present disclosure. For example, the configuration of one embodiment can be added to the configuration of another embodiment, and part of the configuration of one embodiment can be replaced with the configuration of another embodiment. Furthermore, part of the configuration of one embodiment can be deleted.

In the massage machine 1 of the above embodiment, a manipulation performed in the test treatment (test manipulation) is determined based on the communication between the control part 2 and the user through the voice output part 3 and the voice input part 4 (first catching), but the configuration is not limited to this. For example, the configuration can be such that the control unit 2 obtains data regarding the user's activities and/or physical conditions, such as the distance traveled (route traveled), the number of steps, changes in heart rate, changes in body temperature, etc., on the day from Smart Watch worn by the user, and the manipulations to be performed in the test treatments are determined from this data, or the manipulations to be performed in the test treatments are determined from this data and the communication (first catching).

In the massage machine 1 of this embodiment, the control part 2 creates a treatment course by selecting a certain massage course, based on the test manipulations identified (extracted) at the second hearing, from a plurality of massage courses (in the examples of the above embodiment, 1. Whole body course, 2. Upper body course, 3. Lower body course, 4. Whole body stretch course, 5. Lower body focus course, 6. Whole body fatigue recovery course, 7. Soft course) in the course menu CM stored in the storing part M. However, the configuration is not limited to this.

For example, the configuration can be such that the control part 2 selects, in Step S7 for determining the treatment course, a certain massage manipulation or certain message manipulations from a plurality of massage manipulations stored in the storing part M based on the second catching (communication for catching the result), and creates (composes) a treatment course by the massage manipulation(s) thus selected. According to this configuration, a treatment course is created based on the massage manipulation(s) selected according to the physical conditions of the user, so that treatment that is more suitable for the user can be applied.

In the creation of this treatment course, in the case where, for example, when test manipulations which was effective at the second catching, and treatment areas by the identified manipulations are close to each other, a plurality of treatment areas are combined into one group to create a broad treatment area to which the treatment can be applied. Further, from the manipulations identified in the second catching, it is determined whether the stiff area of the user is the upper body or the lower body, and further, it is determined which of the impact, stretch, and air makes the user feel comfortable, so that a treatment course can be created based on these determinations.

The massage machine 1 can be configured such that the storing part M stores a plurality of massage manipulations, and at least one massage course (basic set) composed of a combination of certain massage manipulations, and the control part 2 creates (composes) a treatment course based on the plurality of massage manipulations and at least one massage course stored in the storing part M. According to this configuration, it is possible to create (compose) a treatment course by deleting or adding a massage manipulation from/to the massage course, and thus suppress the processing amount of data or the like in determination of the treatment course. Thereby, the treatment course suitable for the user can be obtained in a short period of time. The treatment course created based on this massage course is determined separately depending on the treatment area, the impact, the stretch, and the air.

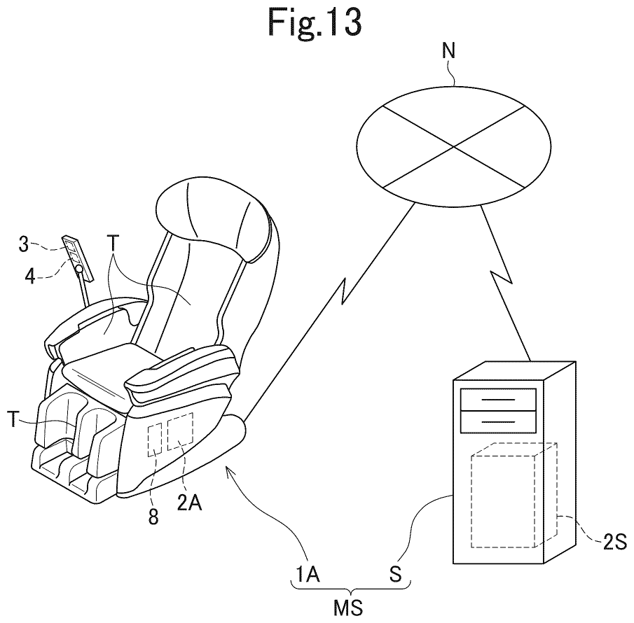

The massage machine 1 of the above embodiment includes the built-in configuration (control part 2) that creates the treatment course suitable for the user without limitation thereto. For example, as shown in FIG. 13, the configuration can be such that a server S is arranged separately from a massage machine 1A, and the treatment course is created by the server S. That is, the configuration can be such that a massage system MS is formed by the massage machine 1A and the server S connected to the massage machine 1A via a network N.

Specifically, the massage system MS includes: the massage machine 1A including treatment parts T for treating a plurality of treatment target portions of the user, a voice output part 3, a voice input part 4, a machine control part 2A to which the treatment parts T, the voice output part 3, and the voice input part 4 are connected, and a communication part 8; and the server S including a server control part 2S and being able to communicate with the massage machine 1A. The machine control part 2A is configured to perform Step S5 of performing test manipulations respectively to the plurality of treatment target portions of the user using the treatment parts T (performing test manipulations), and catching a result of the test manipulations by communicating with the user (second catching) through the voice output part 3 and the voice input part 4 of the massage machine 1A during or after the test treatment, and Step S7 of creating (determining) a treatment course composed of a combination of a plurality of massage manipulations by the treatment parts T based on the result of the test manipulations (based on the second catching). That is, in the massage system MS, the machine control part 2A controls only the movements of the respective elements (e.g., treatment parts T) of the massage machine 1A, and the server control part 2S performs various processes of Step S2 to Step S9 in the flow shown in FIG. 3 (execution of the first catching and the second catching, extracting a keyword and identifying a manipulation which was effective, creating a treatment course, etc.) and commanding to the machine control part 2A based on the result of the processes. The server S can be a physical server, or a virtual server installed on a cloud platform. Further, the communication part 8 of the massage machine 1A is connected to the network N via WiFi or Bluetooth.

In this configuration, too, the result of the test manipulations to the plurality of treatment targets by the treatment parts T (e.g., comfort with the test manipulations applied to the respective treatment target portions) is caught by communicating with the user at the second catching, and the treatment course is determined based on the content caught by the communication with the user.

Specific configurations of the test manipulations or treatment courses are not limited to the above manipulations or courses, and can be subjected to various modifications.

What is claimed is:

1. A massage machine comprising treatment parts for treating a plurality of treatment target portions of a user, a voice output part, a voice input part, and a control part to which the treatment parts, the voice output part, and the voice input part are connected,
the control part performing:
a step of performing test manipulations using the treatment parts respectively to the plurality of target portions of the user;
a step of catching a result of the test treatments by communicating with the user through the voice output part and the voice input part; and
a step of determining a treatment course composed of a plurality of a combination of massage manipulations by the treatment parts based on the result of the test treatments caught by the communication with the user.

2. The massage machine according to claim 1, wherein the control part performs, before performing the test treatments, a step of performing communication for test preparation for catching at least one of activities and conditions of the user by communicating with the user through the voice output part and the voice input part, and determines a treatment target portion to which each corresponding one of the test treatments is performed based on the result of the communication for test preparation.

3. The massage machine according to claim 1, further comprising a storing part that is connected to the control part and stores a plurality of massage manipulations be respectively performed by the treatment parts, wherein a treatment course is composed of the massage manipulations stored in the storing part.

4. The massage machine according to claim 3, wherein in the step of determining the treatment course, the control part selects a certain massage manipulation or certain massage manipulations from the plurality of massage manipulations stored in the storing part based on the result of the test treatments caught by the communication with the user, and composes the treatment course by the selected massage manipulation or massage manipulations.

5. The massage machine according to claim 4, wherein the storing part stores at least one basic set composed of a combination of certain massage manipulations, and
the control part composes the treatment course based on the plurality of massage manipulations and the at least one basic set stored in the storing part.

6. A massage system comprising: a massage machine comprising treatment parts for treating a plurality of treatment target portions of a user, a voice output part, a voice input part, a machine control part to which the treatment parts, the voice output part, and the voice input part are connected, and a communication part; and a server comprising a server control part and being able to communicate with the massage machine, wherein
the machine control part is configured to perform test treatments respectively to the plurality of treatment target portions of the user using the treatment parts, and
the server control part is configured to perform a step of catching a result of the test treatments by communicating with the user through the voice output part and the voice input part of the massage machine, and a step of determining a treatment course composed of a combination of a plurality of massage manipulations by the treatment parts based on the result of the test treatments caught by the communication with the user.
